(19) United States
(12) Patent Application Publication
(10) Pub. No.: US 2016/0166786 A1
(43) Pub. Date: Jun. 16, 2016

(54) SYSTEMS, DEVICES, AND METHODS INCLUDING PERSONAL VAPORIZING INHALERS HAVING CARTRIDGES CONFIGURED TO HOLD MULTIPLE UNIT DOSES

(71) Applicant: Craig E. Kinzer, Issaquah, WA (US)

(72) Inventor: Craig E. Kinzer, Issaquah, WA (US)

(21) Appl. No.: 14/968,689

(22) Filed: Dec. 14, 2015

Related U.S. Application Data
(60) Provisional application No. 62/092,259, filed on Dec. 16, 2014.

(51) Int. Cl. A61M 15/00; A61K 31/352; A61K 31/045; A61K 9/00; A61K 31/122; A61K 31/575; A61K 31/7016; A61M 11/04; A61K 31/015

(52) U.S. Cl. CPC A61M 15/0066; A61M 11/042; A61M 15/0003; A61K 31/352; A61K 31/045; A61K 31/015; A61K 31/122; A61K 31/575; A61K 31/7016; A61K 9/0078; A61M 2205/35; A61M 2205/33

(57) ABSTRACT

Systems, Devices, and Methods are described that enable users to manage, receive, utilize, and the like cannabis related services and products. Also described are systems, devices, and methods including an electronic vaporizer device having a cartridge assembly including one or more cartridges, each cartridge configured to hold multiple unit doses of an active agent; a cannabinoid activation component; and at least one of a terpenoid activation component or a flavonoid activation component. Also described are systems, devices, and methods for managing treatments associated with phyto-cannabinoid unit dose forms for treating various diseases or disorders.

### PRIORITY CLAIM

This application claims the benefit of U.S. Provisional Application No. 62/092,259, filed on Dec. 16, 2014, which is incorporated by reference herein in its entirety.

### SUMMARY

In an aspect, the present disclosure is directed to, among other things, an electronic vaporizer device. In an embodiment, the electronic vaporizer device includes a cartridge assembly having one or more cartridges. In an embodiment, the cartridge assembly includes at least one cartridge having multiple unit doses of an active agent. In an embodiment, each cartridge is configured to hold multiple unit doses of a composition including at least one of a cannabinoid, a terpenoid, or a flavonoid, or combinations or mixtures thereof. In an embodiment, each cartridge is configured to hold multiple unit doses of a vaporizable composition including at least one of a cannabinoid, a terpenoid, or a flavonoid, or combinations or mixtures thereof. In an embodiment, each cartridge is configured to hold multiple unit doses of an aerosolable composition including at least one of a cannabinoid, a terpenoid, or a flavonoid, or combinations or mixtures thereof. In an embodiment, the cartridge assembly includes at least one cartridge having multiple unit doses of a solid plant extract composition including at least one of a cannabinoid, a terpenoid, or a flavonoid, or combinations or mixtures thereof.

In an embodiment, the electronic vaporizer device includes a cannabinoid activation component. In an embodiment, the electronic vaporizer device includes a terpenoid activation component. In an embodiment, the electronic vaporizer device includes a flavonoid activation component.

In an embodiment, the electronic vaporizer device includes a communication interface component.

In an aspect, the present disclosure is directed to, among other things, a vaporizing inhaler device. In an embodiment, the vaporizing inhaler device includes an aerosol generation assembly including one or more active agent reservoirs forming part of a modular structure. In an embodiment, the modular structure includes an active agent reservoir having multiple unit doses of a vaporizable composition including at least one of a cannabinoid, a terpenoid, or a flavonoid.

In an embodiment, the vaporizing inhaler device includes a vaporization chamber. In an embodiment, the vaporizing inhaler device includes a cannabinoid activation component operably coupled to the vaporization chamber. In an embodiment, the vaporizing inhaler device includes a terpenoid activation component operably coupled to the vaporization chamber. In an embodiment, the vaporizing inhaler device includes a flavonoid activation component operably coupled to the vaporization chamber.

In an embodiment, the vaporizing inhaler device includes a communication interface.

In an embodiment, the vaporizing inhaler device includes a target experience component operably coupled to the aerosol generation assembly. In an embodiment, the target experience component is operable to generate one or more control commands for formulating a target aerosol composition having a target cannabinoid content, a target terpenoid content, or a target flavonoid content. In an embodiment, the vaporizing inhaler device includes a target experience component operably coupled to the aerosol generation assembly. In an embodiment, the target experience component is operable to generate one or more control commands for formulating a target aerosol composition having a target unit dose based on a pain mitigation profile or a stress mitigation profile. In an embodiment, the target experience component is operable to generate one or more control commands for formulating a target aerosol composition having a target unit dose based one or more communication from a physiological sensor device, a wearable sensor, exercise tracker or the like. In an embodiment, the target experience component is operable to generate one or more control commands for formulating a target aerosol composition having a target unit dose based one or more inputs from a biometric authorization component.

In an embodiment, the target experience component is operable to generate one or more control commands for formulating a target aerosol composition having a target unit dose based on an auto-immune disease profile. In an embodiment, the target experience component is operable to generate one or more control commands for formulating a target aerosol composition having a target unit dose based on a target cannabinoid/terpenoid/flavonoid profile. In an embodiment, the target experience component is operable to generate one or more control commands for formulating a target aerosol composition having a target unit dose based on a cannabis experience profile.

In an embodiment, the vaporizing inhaler device includes an authorization device including at least one biometric interface. In an embodiment, the vaporizing inhaler device includes an authorization device including at least one mechanical lock. In an embodiment, the vaporizing inhaler device includes an authorization circuit including a speech recognition component.

### BRIEF DESCRIPTION OF THE FIGURES

FIG. 1A is a perspective view of an electronic vaporizer device according to an embodiment.

FIG. 1B is a perspective view of unit doses according to an embodiment.

FIG. 1C is a perspective view of an electronic vaporizer device to an embodiment.

FIG. 2A is a perspective view of a vaporizing inhaler device according to an embodiment.

FIG. 2B is a perspective view of a vaporizing inhaler device according to an embodiment.

### DETAILED DESCRIPTION

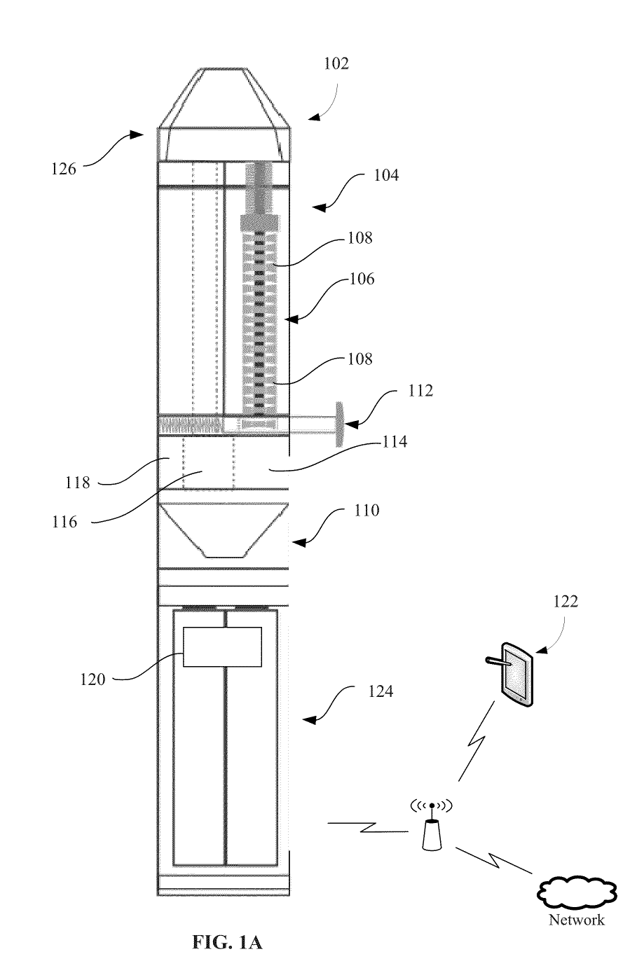

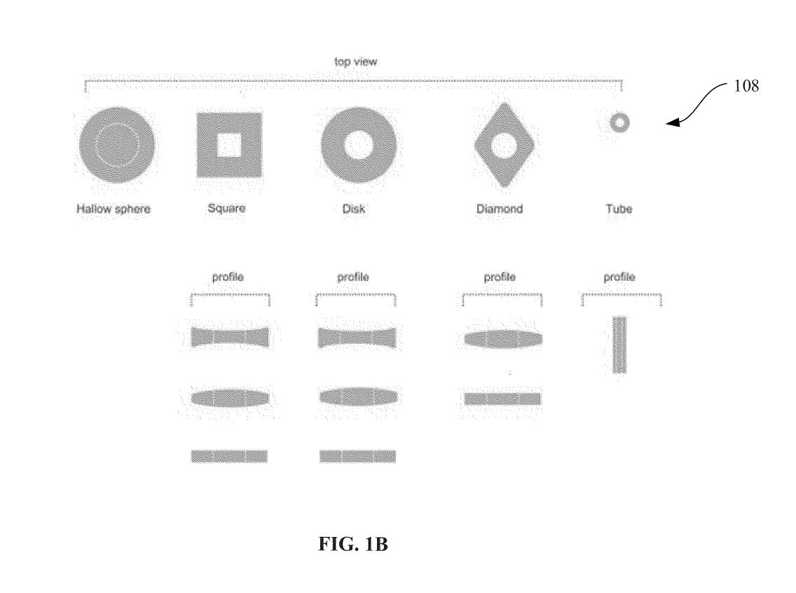

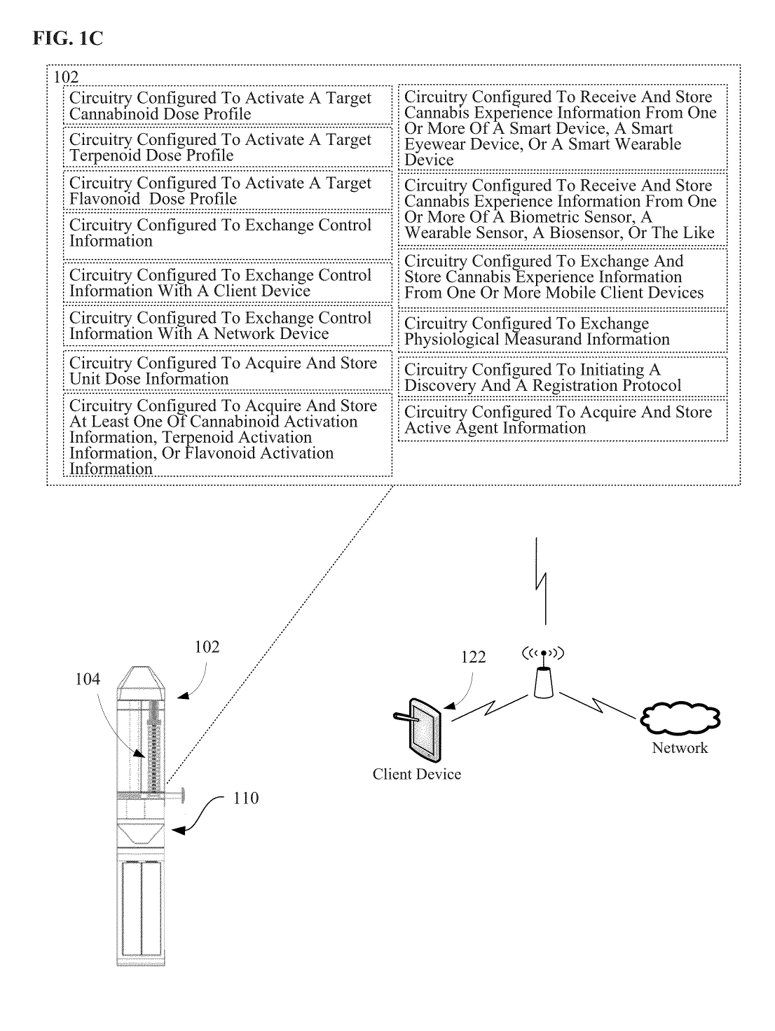

FIGS. 1A and 1C show an electronic vaporizer device 102 in which one or more methodologies or technologies can be implemented, for example, to treat cannabinoid receptor-mediated diseases or disorders, manage a cannabis experience, manage a pain mitigation regimen, manage a stress mitigation regimen, or the like.

In an embodiment, one or more of the methodologies or technologies can be implemented to treat cannabinoid receptor-mediated diseases or disorders of the central nervous system (CNS). Diseases or disorders of the central nervous system include, among others, depression, anxiety, attention deficit hyperactivity disorder (ADHD) and the like. Further CNS diseases or disorders include ulcerative colitis; disorders where increased angiogenesis may be beneficial (e.g., diabetes, gangrene, or the like); disorders in which a lack of dopamine or serotonin is involved; disorders in which improved cognition may be beneficial (e.g., Alzheimer's disease, Parkinson's disease, schizophrenia, or the like); Tourette's Syndrome; nausea, vomiting, anorexia nervosa, spasticity, major depressive disorder, cachexia, wasting syndromes, appetite suppression, glaucoma, epilepsy, Dravet Syndrome, multiple sclerosis, asthma, and pain, including pain involved with cancer, HIV, migraines and generalized neuropathic pain. In an embodiment, one or more of the disclosed methodologies or technologies can be implemented to treat a disease or disorder that elicits a therapeutic response in a patient using an active agent such as a cannabinoid, a terpenoid, a flavonoid or the like.

In an embodiment, the electronic vaporizer device 102 includes a cartridge assembly 104 including one or more cartridges 106. In an embodiment, a cartridge 106 is configured to hold multiple unit doses 108 of an active agent.

Referring to FIG. 1B, in an embodiment, the shape of the unit dose 108 can be a geometrical shape including and regular geometric shapes, such as circular, hexagonal, pentagonal, rectangular, triangular, and the like, as well as irregular geometric shapes. In an embodiment, each unit doses 108 comprises from about 0.1 milligrams to about 500 milligrams of at least one active agent. In an embodiment, each unit doses 108 comprises from about 0.5 milligrams to about 250 milligrams of at least one active agent. In an embodiment, each unit doses 108 comprises from about 1 milligrams to about 100 milligrams of at least one active agent.

Non-limiting examples of active agents include one or more cannabinoids. Further non-limiting examples of cannabinoids includes those found naturally in cannabis or members of the Cannabis species (e.g. phyto-cannabinoids, phyto-cannabichromenes, phyto-cannabidiols, phyto-cannabidiolic acids, phyto-cannabigerols, phyto-cannabinols, phyto-cannabidivarins, phyto-tetrahydrocannabinolic acids, phyto-tetrahydrocannabivarins, and the like), including Cannabis sativa, Cannabis indica, and Cannabis ruderalis, and chemovars, cultivars, genetic crosses, self-crosses and hybrids thereof. Further non-limiting examples of active agents include synthetic cannabinoids and human cannabinoids (i.e., endocannabinoids), including nabilone, dronabinol, and rimonabant. Further non-limiting examples of active agents include cannabidiols, cannabigerols, cannabichromenes, cannabinols, and the like

Further non-limiting examples of active agents include Δ⁹-tetrahydrocannabinol; Δ⁹-tetrahydrocannabiorcol; Δ⁹-tetrahydrocannabivarin; 10-O-ethylcannabitriol; 6a,7,10a-trihydroxytetrahydrocannabinol; 7,8-dehydro-10-O-ethylcannabitriol; 9,10-epoxycannabitriol; cannabichromene; cannabicitran; cannabicyclol; cannabidiol; cannabidivarin; cannabielsoin; cannabigerol; cannabinol; dihydrocannabinol; and the like, and analogues and derivatives thereof. See e.g., Ross et al., Phytochem Anal, January-February; 16(1):45-(2005). Further non-limiting examples of active agents include Δ⁹ tetrahydrocannabinol, Δ⁸ tetrahydrocannabinol, cannabidiol, cannabigerol, cannabichromene, cannabinol, and the like, and analogues and derivatives thereof, including ether, ester and amide derivatives. Further non-limiting examples of active agents include phyto-cannabinoids (THC), phyto-cannabichromenes (CBC), phyto-cannabidiols (CBD), phyto-cannabidiolic acids (CBD-A), phyto-cannabigerols (CBG), phyto-cannabinols (CBN), phyto-cannabidivarins (CBDV), phyto-tetrahydrocannabinolic acids (THC-A), phyto-tetrahydrocannabivarins (THCV), and the like.

Further non-limiting examples of active agents include one or more terpenoids. Non-limiting examples of terpenoids include borneol, β-caryophyllene, cineole, delta-3-carene, limonene, D-linalool, β-myrcene, pinene, pulegone, sabinene, terpineol, and the like.

Further non-limiting examples of active agents include one or more flavonoids. Non-limiting examples of flavonoids include apigenin, quercetin, cannflavin A, B-sitosterol, and the like. Further non-limiting examples of flavonoids include flavonoid glycosides (e.g., kaempferol 3-O-sphoroside, quercetin 3-O-sophoroside, etc.).

Further non-limiting examples of active agents include those described in U.S. provisional patent application Nos. 62/027,374 and 62/027,391, which are incorporated herein by reference in full.

In an embodiment, the cartridge assembly 104 includes at least one cartridge 106 having multiple unit doses 108 of an inhalable active agent. In an embodiment, the cartridge assembly 104 includes at least one cartridge 106 having multiple unit doses 108 of a vaporizable active agent. In an embodiment, the cartridge assembly 104 includes at least one cartridge 106 having multiple unit doses 108 of an aerosolable active agent. In an embodiment, the cartridge assembly 104 includes at least one cartridge 106 having multiple unit doses 108 of a solid plant extract composition including at least one of a cannabinoid, a terpenoid, or a flavonoid, or combinations or mixtures thereof.

In an embodiment, each unit dose 108 of the active agent comprises a composition including at least one of a cannabinoid, a terpenoid, or a flavonoid, or combinations or mixtures thereof, and a pharmaceutical acceptable carrier.

In an embodiment, the cartridge assembly 104 includes at least one cartridge 106 having multiple unit doses 108 of a vaporizable plant extract composition including at least one of a cannabinoid, a terpenoid, or a flavonoid. In an embodiment, the cartridge assembly 104 includes at least one cartridge 106 having multiple unit doses 108 of an aerosolable plant extract composition including at least one of a cannabinoid, a terpenoid, or a flavonoid. In an embodiment, the cartridge assembly 104 includes at least one cartridge 106 having multiple unit doses 108 of a vaporizable cannabinoid composition.

In an embodiment, the cartridge assembly 104 includes at least one cartridge 106 having multiple unit doses 108 of an aerosolable cannabinoid composition. In an embodiment, the cartridge assembly 104 includes at least one cartridge 106 having multiple unit doses 108 of an inhalable cannabinoid. In an embodiment, the cartridge assembly 104 includes at least one cartridge 106 having multiple unit doses 108 of an inhalable phyto-cannabinoid. In an embodiment, the cartridge assembly 104 includes at least one cartridge 106 having multiple unit doses 108 of an inhalable terpenoid. In an embodiment, the cartridge assembly 104 includes at least one cartridge 106 having multiple unit doses 108 of an inhalable flavonoid.

In an embodiment, the cartridge assembly 104 includes at least one cartridge 106 having multiple solid unit doses 108 of a plant extract composition including at least one cannabinoid, at least one terpenoid, and at least one flavonoid. In an embodiment, the cartridge assembly 104 includes at least one cartridge 106 having multiple solid unit doses 108 of an inhalable composition including at least one cannabinoid, at least one terpenoid, and at least one flavonoid. In an embodiment, the cartridge assembly 104 includes at least one cartridge 106 configured to hold multiple solid unit doses 108 of an inhalable active agent. In an embodiment, the cartridge assembly 104 includes at least a first cartridge and a second cartridge, the second cartridge having a unit dose of an active agent different from a unit dose of the active agent in the first cartridge.

In an embodiment, the electronic vaporizer device 102 includes an activation chamber 110. In an embodiment, the cartridge assembly 104 includes a dispensing assembly 112. In an embodiment, the dispensing assembly 112 is operable to dispense at least one unit dose 108 of an active agent into the activation chamber 110. In an embodiment, the dispensing assembly 112 is operable to dispense at least one solid unit doses 108 of an active agent into the activation chamber 110. In an embodiment, the dispensing assembly 112 is operable to dispense at least one vaporizable unit doses 108 of an active agent into the activation chamber 110.

In an embodiment, the electronic vaporizer device 102 includes a cannabinoid activation component 114. In an embodiment, the activation chamber 110 is operably coupled to the cannabinoid activation component 114.

In an embodiment, the cannabinoid activation component 114 is configured to selectively heat an interior environment within the activation chamber 110 to a temperature and for a duration sufficient to cause the release of one or more cannabinoids from a unit dose received within the activation chamber 110. For example, in an embodiment, the cannabinoid activation component 114 includes circuitry configured to activate a target cannabinoid dose profile.

In an embodiment, circuitry includes, among other things, one or more computing devices such as a processor (e.g., a microprocessor), a central processing unit (CPU), a digital signal processor (DSP), an application-specific integrated circuit (ASIC), a field programmable gate array (FPGA), or the like, or any combinations thereof, and can include discrete digital or analog circuit elements or electronics, or combinations thereof. In an embodiment, circuitry includes one or more ASICs having a plurality of predefined logic components. In an embodiment, circuitry includes one or more FPGA having a plurality of programmable logic components.

In an embodiment, the electronic vaporizer device 102 includes circuitry having one or more components operably coupled (e.g., communicatively, electromagnetically, magnetically, ultrasonically, optically, inductively, electrically, capacitively coupled, or the like) to each other. In an embodiment, circuitry includes one or more remotely located components. In an embodiment, remotely located components are operably coupled via wireless communication. In an embodiment, remotely located components are operably coupled via one or more receivers, transceivers, or transmitters, or the like.

In an embodiment, circuitry includes one or more memory devices that, for example, store instructions or data. For example, in an embodiment, the electronic vaporizer device 102 includes one or more memory devices that store cannabis experience information, cannabis management information, and the like. Non-limiting examples of one or more memory devices include volatile memory (e.g., Random Access Memory (RAM), Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like), persistent memory, or the like. Further non-limiting examples of one or more memory devices include Erasable Programmable Read-Only Memory (EPROM), flash memory, or the like. The one or more memory devices can be coupled to, for example, one or more computing devices by one or more instructions, data, or power buses. In an embodiment, the electronic vaporizer device 102 includes one or more memory device that stores, for example, information regarding user-specific terpene/terpenoid/CBD/THC information, user-specific flavor profile information, user-specific auto-immune diseases information, user-specific pain mitigation profile information, user-specific stress mitigation profile information, user-specific desired feeling/results profile information, and the like. In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, circuitry includes one or more user input/output components that are operably coupled to at least one computing device to control (electrical, electromechanical, software-implemented, firmware-implemented, or other control, or combinations thereof) at least one parameter associated with, for example,

In an embodiment, circuitry includes a computer-readable media drive or memory slot that is configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as a magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., receiver, transceiver, or transmitter, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In an embodiment, the electronic vaporizer device 102 includes circuitry having one or more modules optionally operable for communication with one or more input/output components that are configured to relay user output and/or input. In an embodiment, a module includes one or more instances of electrical, electromechanical, software-implemented, firmware-implemented, or other control devices. Such devices include one or more instances of memory, computing devices, antennas, power or other supplies, logic modules or other signaling modules, gauges or other such active or passive detection components, piezoelectric transducers, shape memory elements, micro-electro-mechanical system (MEMS) elements, or other actuators.

In an embodiment, the cannabinoid activation component 114 includes circuitry including at least one heating element configured to heat an interior environment within the activation chamber 110 to a temperature ranging from about 120° C. to about 220° C. In an embodiment, the cannabinoid activation component 114 includes circuitry including at least one heating element configured to heat an interior environment within the activation chamber 110 to a temperature ranging from about 185° C. to about 220° C.

In an embodiment, the cannabinoid activation component 114 includes circuitry including at least one heating element configured to heat an interior environment within the activation chamber 110 to a temperature of about 185° C. In an embodiment, the cannabinoid activation component 114 includes circuitry including at least one heating element configured to heat an interior environment within the activation chamber 110 to a temperature of about 175° C. In an embodiment, the cannabinoid activation component 114 includes circuitry including at least one heating element configured to heat an interior environment within the activation chamber 110 to a temperature ranging from about 157° C. to about 160° C. In an embodiment, the cannabinoid activation component 114 includes circuitry including at least one heating element configured to heat an interior environment within the activation chamber 110 to a temperature of about 220° C.

In an embodiment, the electronic vaporizer device 102 includes a terpenoid activation component 116. In an embodiment, the terpenoid activation component 116 is configured to selectively heat an interior environment within the activation chamber 110 to a temperature and for a duration sufficient to cause the release of one or more terpenoids from a unit dose received within the activation chamber 110. In an embodiment, the terpenoid activation component 116 includes circuitry configured to activate a target terpenoid dose profile. In an embodiment, the terpenoid activation component 116 includes circuitry including at least one heating element configured to heat an interior environment within the activation chamber 110 to a temperature ranging from about 168° C. to about 224° C.

In an embodiment, the electronic vaporizer device 102 includes a flavonoid activation component 118. In an embodiment, the terpenoid activation component 116 is configured to selectively heat an interior environment within the activation chamber 110 to a temperature and for a duration sufficient to cause the release of one or more flavonoids from a unit dose received within the activation chamber 110. For example, in an embodiment, the flavonoid activation component 118 includes circuitry configured to activate a target flavonoid dose profile. In an embodiment, the flavonoid activation component 118 includes circuitry including at least one heating element configured to heat an interior environment within the activation chamber 110 to a temperature ranging from about 178° C. to about 182° C.

In an embodiment, the electronic vaporizer device 102 includes a communication interface component 120. In an embodiment, the communication interface component 120 includes circuitry configured to exchange control information with a client device 122. In an embodiment, the communication interface component 120 includes circuitry configured to exchange control information with a network device.

In an embodiment, the communication interface component 120 includes circuitry configured to acquire and store unit dose information. In an embodiment, the communication interface component 120 includes circuitry configured to acquire and store at least one of cannabinoid activation information, terpenoid activation information, or flavonoid activation information. In an embodiment, the communication interface component 120 includes circuitry configured to acquire and store active agent information. In an embodiment, the active agent information includes at least one of cannabinoid information, terpenoid information, or flavonoid information.

In an embodiment, the active agent information includes at least one of dose profile information or flavor profile information. In an embodiment, the communication interface component 120 includes circuitry configured to receive and store cannabis experience information from one or more of a smart device, a smart eyewear device, or a smart wearable device. In an embodiment, the communication interface component 120 includes circuitry configured to exchange and store cannabis experience information from one or more of a cell phone device, a computer device, a desktop computer device, a laptop computer device, a managed node device, a notebook computer device, a remote controller, a tablet device, a wearable device, or an application interface with a smart device. In an embodiment, the communication interface component 120 includes circuitry configured to receive and store cannabis experience information from one or more of a biometric sensor, a wearable sensor, a biosensor, or the like.

In an embodiment, the communication interface component 120 includes circuitry configured to exchange and store cannabis experience information from one or more mobile client devices 122. In an embodiment, the communication interface component 120 includes circuitry configured to exchange and store cannabis experience information from one or more client devices 122. In an embodiment, the communication interface component 120 includes circuitry configured to negotiate an authorization protocol and to exchange cannabis experience information with a client device 122.

In an embodiment, the communication interface component 120 includes circuitry configured to exchange cannabis experience information with a client device 122. In an embodiment, the communication interface component 120 includes circuitry configured to exchange cannabis experience information with a client device 122. In an embodiment, the communication interface component 120 includes circuitry configured to exchange cannabis management information with a wearable device, a wearable network device, a wearable sensor, or the like.

In an embodiment, the communication interface component 120 includes circuitry configured to exchange physiological measurand information with a client device 122. In an embodiment, the communication interface component 120 includes circuitry configured to initiating a discovery and a registration protocol that allows a client device 122 and the electronic vaporizer device 102 to find each other and negotiate one or more pre-shared keys.

In an embodiment, the activation chamber 110 is operably coupled to the cannabinoid activation component 114 and the at least one of the terpenoid activation component 116 or the flavonoid activation component 118.

In an embodiment, at least one of the cannabinoid activation component 114, the terpenoid activation component 116, or the flavonoid activation component 118 is configured to activate heating of an interior environment with an activation chamber 110 to a temperature and for a duration sufficient to cause the release of one or more active agents from a unit dose received within the activation chamber 110, responsive to one or more inputs indicative of a target cannabinoid/terpenoid/flavonoid profile.

In an embodiment, at least one of the cannabinoid activation component 114, the terpenoid activation component 116, or the flavonoid activation component 118 is configured to activate heating of an interior environment with an activation chamber 110 to a temperature and for a duration sufficient to cause the release of one or more active agents from a unit dose received within the activation chamber 110, responsive to one or more inputs indicative of a user-specific cannabis experience profile.

In an embodiment, at least one of the cannabinoid activation component 114, the terpenoid activation component 116, or the flavonoid activation component 118 is configured to activate heating of an interior environment with an activation chamber 110 to a temperature and for a duration sufficient to cause the release of one or more active agents from a unit dose received within the activation chamber 110, responsive to one or more inputs indicative of a user-specific auto-immune disease profile.

In an embodiment, at least one of the cannabinoid activation component 114, the terpenoid activation component 116, or the flavonoid activation component 118 is configured to activate heating of an interior environment with an activation chamber 110 to a temperature and for a duration sufficient to cause the release of one or more active agents from a unit dose received within the activation chamber 110, responsive to one or more inputs indicative of a user-specific pain mitigation profile.

In an embodiment, at least one of the cannabinoid activation component 114, the terpenoid activation component 116, or the flavonoid activation component 118 is configured to activate heating of an interior environment with an activation chamber 110 to a temperature and for a duration sufficient to cause the release of one or more active agents from a unit dose received within the activation chamber 110, responsive to one or more inputs indicative of a user-specific stress mitigation profile.

In an embodiment, at least one of the cannabinoid activation component 114, the terpenoid activation component 116, or the flavonoid activation component 118 is configured to activate heating of an interior environment with an activation chamber 110 to a temperature and for a duration sufficient to cause the release of one or more active agents from a unit dose received within the activation chamber 110, responsive to one or more inputs indicative of a real-time physiological measurand.

In an embodiment, the electronic vaporizer device 102 includes a power supply 124 operably coupled to one or more components. In an embodiment, the electronic vaporizer device 102 includes mouthpiece assembly 126.

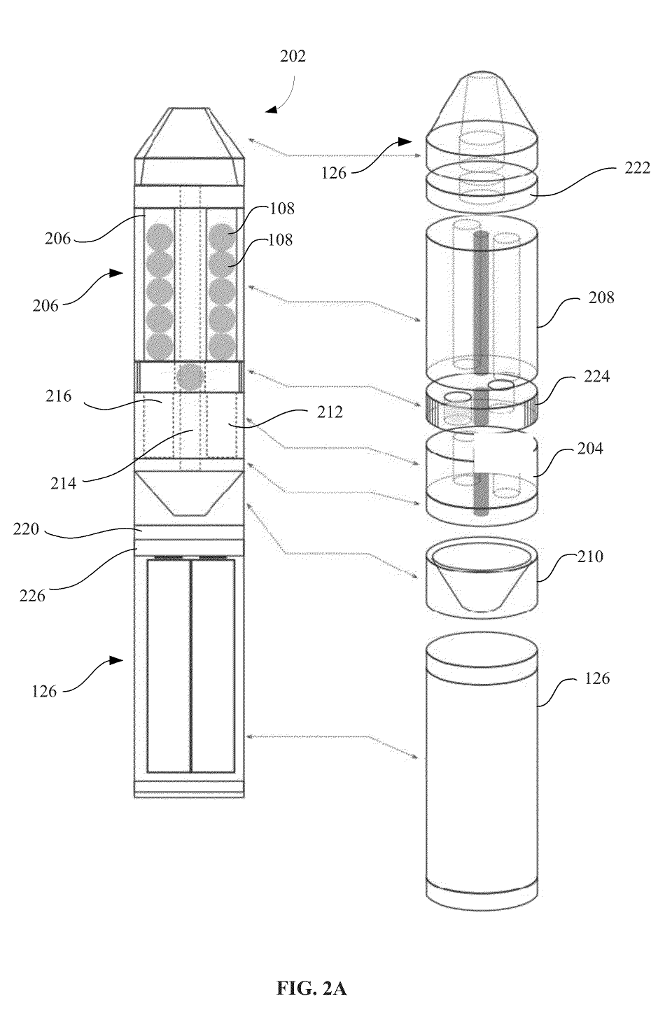

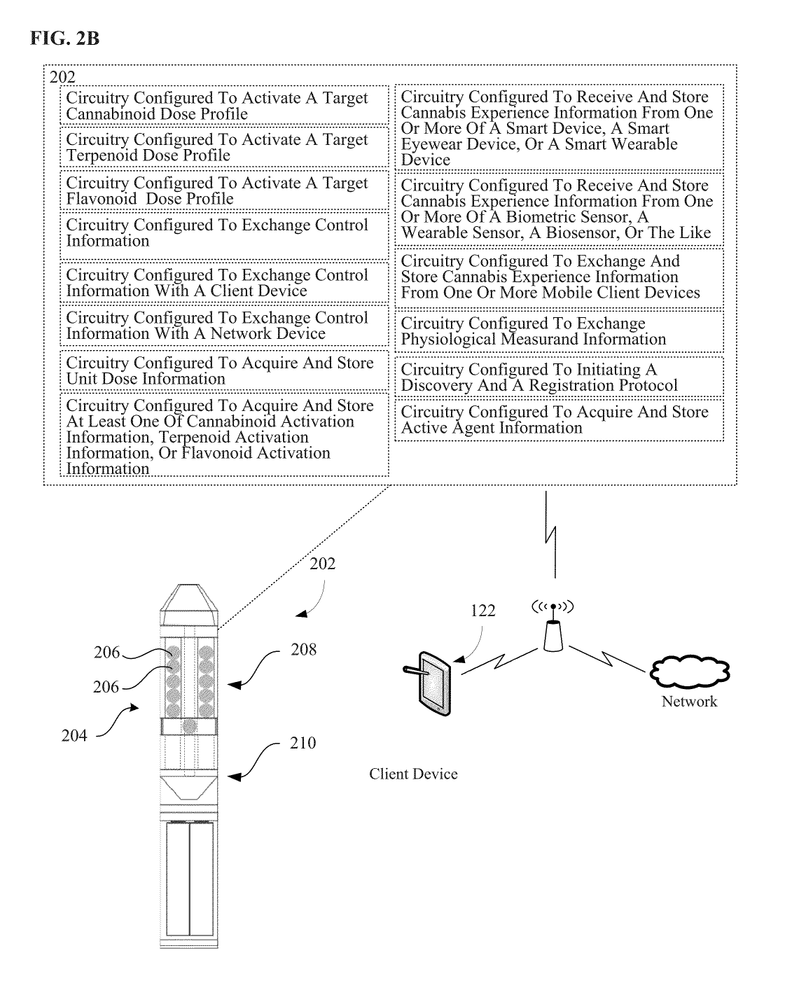

FIGS. 2A and 2B show a vaporizing inhaler device 202 in which one or more methodologies or technologies can be implemented, for example, to treat cannabinoid receptor-mediated diseases or disorders, manage a cannabis experience, manage a pain mitigation regimen, manage a stress mitigation regimen, or the like.

In an embodiment, the vaporizing inhaler device 202 includes an aerosol generation assembly 204 including one or more active agent reservoirs 206 forming part of a modular structure 208. In an embodiment, the aerosol generation assembly 204 is configured to generate an aerosol from an active agent composition fed from the one or more active agent reservoirs 206. For example, in an embodiment, the aerosol generation assembly 204 includes at least one nozzle for generating an aerosol. In an embodiment, the aerosol generation assembly 204 is configured to vaporize an active agent composition fed from the one or more active agent reservoirs 206 by applying heat to the active agent composition.

In an embodiment, the vaporizing inhaler device 202 includes a vaporization chamber 210. In an embodiment, the vaporizing chamber 210 is operably coupled to at least one of the one or more reservoirs 206. In an embodiment, the vaporizing chamber 210 is in fluidic communication with at least one of the one or more reservoir 206.

In an embodiment, the vaporizing inhaler device 202 includes a cannabinoid activation component 212. In an embodiment, the cannabinoid activation component 212 includes one or more heating elements. In an embodiment, the one or more heating elements include at least one of a microwave heating element, an infrared heating element, a thermal heating element, a sonic heating element, an electromagnetic energy heating element, an optical heating element, or an electrical heating element. In an embodiment, the cannabinoid activation component 212 is operably coupled to the vaporization chamber 210.

In an embodiment, the cannabinoid activation component 212 is configured to selectively heat an interior environment within the vaporization chamber 210 to a temperature and for a duration sufficient to cause the release of one or more cannabinoids from a unit dose received within the vaporization chamber 210. For example, in an embodiment, the cannabinoid activation component 212 includes circuitry configured to activate a target cannabinoid dose profile.

In an embodiment, the vaporizing inhaler device 202 includes a terpenoid activation component 214. In an embodiment, the terpenoid activation component 214 includes one or more heating elements. In an embodiment, the one or more heating elements include at least one of a microwave heating element, an infrared heating element, a thermal heating element, a sonic heating element, an electromagnetic energy heating element, an optical heating element, or an electrical heating element. In an embodiment, the terpenoid activation component 214 is operably coupled to the vaporization chamber 210.

In an embodiment, the terpenoid activation component 214 is configured to selectively heat an interior environment within the vaporization chamber 210 to a temperature and for a duration sufficient to cause the release of one or more terpenoids from a unit dose received within the vaporization chamber 210. For example, in an embodiment, the terpenoid activation component 214 includes circuitry configured to activate a terpenoid dose profile.

In an embodiment, the vaporizing inhaler device 202 includes a flavonoid activation component 216. In an embodiment, the flavonoid activation component 216 includes one or more heating elements. In an embodiment, the one or more heating elements include at least one of a microwave heating element, an infrared heating element, a thermal heating element, a sonic heating element, an electromagnetic energy heating element, an optical heating element, or an electrical heating element. In an embodiment, the flavonoid activation component 216 is operably coupled to the vaporization chamber 210.

In an embodiment, the flavonoid activation component 216 includes circuitry configured to activate a flavonoid dose profile. For example, in an embodiment, the flavonoid activation component 216 is configured to selectively heat an interior environment within the vaporization chamber 210 to a temperature and for a duration sufficient to cause the release of one or more flavonoids from a unit dose received within the vaporization chamber 210.

In an embodiment, the vaporizing inhaler device 202 includes a communication interface component 120.

In an embodiment, the modular structure 208 includes at least active agent reservoir 206 including multiple unit doses of a vaporizable composition including at least one of a cannabinoid, a terpenoid, or a flavonoid. In an embodiment, at least one of the one or more active agent reservoirs 206 include multiple unit doses of a vaporizable composition including a plant extract having at least one of a cannabinoid, a terpenoid, or a flavonoid. In an embodiment, at least one of the one or more active agent reservoirs 206 include multiple unit doses of an aerosolable composition including at least one of a cannabinoid, a terpenoid, or a flavonoid.

In an embodiment, at least one of the one or more active agent reservoirs 206 include multiple unit doses of at least one of a vaporizable cannabinoid composition, a vaporizable terpenoid composition, or a vaporizable flavonoid composition. In an embodiment, at least one of the one or more active agent reservoirs 206 include multiple unit doses of at least one of an aerosolable cannabinoid composition, an aerosolable terpenoid composition, an aerosolable flavonoid composition.

In an embodiment, at least one of the one or more active agent reservoirs 206 include at least one humectant such as, for example, propylene glycol, glycerol, and the like.

In an embodiment, the vaporizing inhaler device 202 includes a target experience component 218. In an embodiment, the target experience component 218 is operably coupled to the aerosol generation assembly 204. In an embodiment, the target experience component 218 is operable to generate one or more control commands for formulating a target aerosol composition having a target cannabinoid content, a target terpenoid content, or a target flavonoid content. In an embodiment, the target experience component 218 is operable to generate one or more control commands for formulating a target aerosol composition having a target unit dose based on a pain mitigation profile or a stress mitigation profile.

In an embodiment, the target experience component 218 is operably coupled to the aerosol generation assembly 204. In an embodiment, target experience component 218 is operable to generate one or more control commands for formulating a target aerosol composition having a target unit dose based on an auto-immune disease profile. In an embodiment, the vaporizing inhaler device 202 includes a target experience component 218 operably coupled to the aerosol generation assembly 204, the target experience component 218 operable to generate one or more control commands for formulating a target aerosol composition having a target unit dose based on a target cannabinoid/terpenoid/flavonoid profile. In an embodiment, the target experience component 218 operable to generate one or more control commands for formulating a target aerosol composition having a target unit dose based on a cannabis experience profile. In an embodiment, the target experience component 218 operable to generate one or more control commands for formulating a target aerosol composition having a target unit dose based on one or more inputs from a biometric authorization component. In an embodiment, the target experience component 218 operable to generate one or more control commands for formulating a target aerosol composition having a target unit dose based on one or more inputs from a wearable physiologic sensor.

In an embodiment, the vaporizing inhaler device 202 includes an authorization device 220 including at least one biometric interface 222. In an embodiment, the vaporizing inhaler device 202 includes an authorization device 220 including at least one mechanical lock 224. In an embodiment, the vaporizing inhaler device 220 includes an authorization circuit 226 including a speech recognition component.

The claims, description, and drawings of this application may describe one or more of the instant technologies in operational/functional language, for example as a set of operations to be performed by a computer. Such operational/functional description in most instances can be specifically-configured hardware (e.g., because a general purpose computer in effect becomes a special purpose computer once it is programmed to perform particular functions pursuant to instructions from program software).

Importantly, although the operational/functional descriptions described herein are understandable by the human mind, they are not abstract ideas of the operations/functions divorced from computational implementation of those operations/functions. Rather, the operations/functions represent a specification for the massively complex computational machines or other means. As discussed in detail below, the operational/functional language must be read in its proper technological context, i.e., as concrete specifications for physical implementations.

The logical operations/functions described herein are a distillation of machine specifications or other physical mechanisms specified by the operations/functions such that the otherwise inscrutable machine specifications may be comprehensible to the human mind. The distillation also allows one of skill in the art to adapt the operational/functional description of the technology across many different specific vendors' hardware configurations or platforms, without being limited to specific vendors' hardware configurations or platforms.

Some of the present technical description (e.g., detailed description, drawings, claims, etc.) may be set forth in terms of logical operations/functions. As described in more detail in the following paragraphs, these logical operations/functions are not representations of abstract ideas, but rather representative of static or sequenced specifications of various hardware elements. Differently stated, unless context dictates otherwise, the logical operations/functions are representative of static or sequenced specifications of various hardware elements. This is true because tools available to implement technical disclosures set forth in operational/functional formats—tools in the form of a high-level programming language (e.g., C, java, visual basic), etc.), or tools in the form of Very high speed Hardware Description Language (“VIDAL,” which is a language that uses text to describe logic circuits—)—are generators of static or sequenced specifications of various hardware configurations. This fact is sometimes obscured by the broad term “software,” but, as shown by the following explanation, what is termed “software” is a shorthand for a massively complex interchanging/specification of ordered-matter elements. The term “ordered-matter elements” may refer to physical components of computation, such as assemblies of electronic logic gates, molecular computing logic constituents, quantum computing mechanisms, etc.

For example, a high-level programming language is a programming language with strong abstraction, e.g., multiple levels of abstraction, from the details of the sequential organizations, states, inputs, outputs, etc., of the machines that a high-level programming language actually specifies. See, e.g., High-level Programming Language., Wikipedia. Wikimedia Foundation, 18 Jan. 2014. Web. 4 Feb. 2014. In order to facilitate human comprehension, in many instances, high-level programming languages resemble or even share symbols with natural languages. See, e.g., Natural Language., Wikipedia. Wikimedia Foundation, 14 Jan. 2014. Web. 4 Feb. 2014.

It has been argued that because high-level programming languages use strong abstraction (e.g., that they may resemble or share symbols with natural languages), they are therefore a “purely mental construct” (e.g., that “software”—a computer program or computer—programming—is somehow an ineffable mental construct, because at a high level of abstraction, it can be conceived and understood in the human mind). This argument has been used to characterize technical description in the form of functions/operations as somehow “abstract ideas.” In fact, in technological arts (e.g., the information and communication technologies) this is not true.

The fact that high-level programming languages use strong abstraction to facilitate human understanding should not be taken as an indication that what is expressed is an abstract idea. In an embodiment, if a high-level programming language is the tool used to implement a technical disclosure in the form of functions/operations, it can be understood that, far from being abstract, imprecise, “fuzzy,” or “mental” in any significant semantic sense, such a tool is instead a near incomprehensibly precise sequential specification of specific computational—machines—the parts of which are built up by activating/selecting such parts from typically more general computational machines over time (e.g., clocked time). This fact is sometimes obscured by the superficial similarities between high-level programming languages and natural languages. These superficial similarities also may cause a glossing over of the fact that high-level programming language implementations ultimately perform valuable work by creating/controlling many different computational machines.

The many different computational machines that a high-level programming language specifies are almost unimaginably complex. At base, the hardware used in the computational machines typically consists of some type of ordered matter (e.g., traditional electronic devices (e.g., transistors), deoxyribonucleic acid (DNA), quantum devices, mechanical switches, optics, fluidics, pneumatics, optical devices (e.g., optical interference devices), molecules, etc.) that are arranged to form logic gates. Logic gates are typically physical devices that may be electrically, mechanically, chemically, or otherwise driven to change physical state in order to create a physical reality of Boolean logic.

Logic gates may be arranged to form logic circuits, which are typically physical devices that may be electrically, mechanically, chemically, or otherwise driven to create a physical reality of certain logical functions. Types of logic circuits include such devices as multiplexers, registers, arithmetic logic units (ALUs), computer memory devices, etc., each type of which may be combined to form yet other types of physical devices, such as a central processing unit (CPU)—the best known of which is the microprocessor. A modern microprocessor will often contain more than one hundred million logic gates in its many logic circuits (and often more than a billion transistors). See, e.g., Logic Gates., Wikipedia. Wikimedia Foundation, 2 Apr. 2014. Web. 4 Feb. 2014.

The logic circuits forming the microprocessor are arranged to provide a microarchitecture that will carry out the instructions defined by that microprocessor's defined Instruction Set Architecture. The Instruction Set Architecture is the part of the microprocessor architecture related to programming, including the native data types, instructions, registers, addressing modes, memory architecture, interrupt and exception handling, and external Input/Output. See, e.g., Computer Architecture., Wikipedia. Wikimedia Foundation, 2 Feb. 2014. Web. 4 Feb. 2014.

The Instruction Set Architecture includes a specification of the machine language that can be used by programmers to use/control the microprocessor. Since the machine language instructions are such that they may be executed directly by the microprocessor, typically they consist of strings of binary digits, or bits. For example, a typical machine language instruction might be many bits long (e.g., 32, 64, or 128 bit strings are currently common). A typical machine language instruction might take the form “11110000101011110000111100111111” (a 32 bit instruction).

It is significant here that, although the machine language instructions are written as sequences of binary digits, in actuality those binary digits specify physical reality. For example, if certain semiconductors are used to make the operations of Boolean logic a physical reality, the apparently mathematical bits “1” and “0” in a machine language instruction actually constitute a shorthand that specifies the application of specific voltages to specific wires. For example, in some semiconductor technologies, the binary number “1” (e.g., logical “1”) in a machine language instruction specifies around +5 volts applied to a specific “wire” (e.g., metallic traces on a printed circuit board) and the binary number “0” (e.g., logical “0”) in a machine language instruction specifies around −5 volts applied to a specific “wire.” In addition to specifying voltages of the machines' configuration, such machine language instructions also select out and activate specific groupings of logic gates from the millions of logic gates of the more general machine. Thus, far from abstract mathematical expressions, machine language instruction programs, even though written as a string of zeros and ones, specify many, many constructed physical machines or physical machine states.

Machine language is typically incomprehensible by most humans (e.g., the above example was just ONE instruction, and some personal computers execute more than two billion instructions every second). See, e.g., Instructions per Second., Wikipedia. Wikimedia Foundation, 13 Jan. 2014. Web. 4 Feb. 2014.

Thus, programs written in machine language—which may be tens of millions of machine language instructions long—are incomprehensible. In view of this, early assembly languages were developed that used mnemonic codes to refer to machine language instructions, rather than using the machine language instructions' numeric values directly (e.g., for performing a multiplication operation, programmers coded the abbreviation “mult,” which represents the binary number “011000” in MIPS machine code). While assembly languages were initially a great aid to humans controlling the microprocessors to perform work, in time the complexity of the work that needed to be done by the humans outstripped the ability of humans to control the microprocessors using merely assembly languages.

At this point, it was noted that the same tasks needed to be done over and over, and the machine language necessary to do those repetitive tasks was the same. In view of this, compilers were created. A compiler is a device that takes a statement that is more comprehensible to a human than either machine or assembly language, such as “add 2+2 and output the result,” and translates that human understandable statement into a complicated, tedious, and immense machine language code (e.g., millions of 32, 64, or 128 bit length strings). Compilers thus translate high-level programming language into machine language.

This compiled machine language, as described above, is then used as the technical specification which sequentially constructs and causes the interoperation of many different computational machines such that humanly useful, tangible, and concrete work is done. For example, as indicated above, such machine language—the compiled version of the higher-level language—functions as a technical specification which selects out hardware logic gates, specifies voltage levels, voltage transition timings, etc., such that the humanly useful work is accomplished by the hardware.

Thus, a functional/operational technical description, when viewed by one of skill in the art, is far from an abstract idea. Rather, such a functional/operational technical description, when understood through the tools available in the art such as those just described, is instead understood to be a humanly understandable representation of a hardware specification, the complexity and specificity of which far exceeds the comprehension of most any one human. Accordingly, any such operational/functional technical descriptions may be understood as operations made into physical reality by (a) one or more interchained physical machines, (b) interchained logic gates configured to create one or more physical machine(s) representative of sequential/combinatorial logic(s), (c) interchained ordered matter making up logic gates (e.g., interchained electronic devices (e.g., transistors), DNA, quantum devices, mechanical switches, optics, fluidics, pneumatics, molecules, etc.) that create physical reality representative of logic(s), or (d) virtually any combination of the foregoing. Indeed, any physical object which has a stable, measurable, and changeable state may be used to construct a machine based on the above technical description. Charles Babbage, for example, constructed the first computer out of wood and powered by cranking a handle.

Thus, far from being understood as an abstract idea, it can be recognizes that a functional/operational technical description as a humanly-understandable representation of one or more almost unimaginably complex and time sequenced hardware instantiations. The fact that functional/operational technical descriptions might lend themselves readily to high-level computing languages (or high-level block diagrams for that matter) that share some words, structures, phrases, etc. with natural language simply cannot be taken as an indication that such functional/operational technical descriptions are abstract ideas, or mere expressions of abstract ideas. In fact, as outlined herein, in the technological arts this is simply not true. When viewed through the tools available to those of skill in the art, such functional/operational technical descriptions are seen as specifying hardware configurations of almost unimaginable complexity.

As outlined above, the reason for the use of functional/operational technical descriptions is at least twofold. First, the use of functional/operational technical descriptions allows near-infinitely complex machines and machine operations arising from interchained hardware elements to be described in a manner that the human mind can process (e.g., by mimicking natural language and logical narrative flow). Second, the use of functional/operational technical descriptions assists the person of skill in the art in understanding the described subject matter by providing a description that is more or less independent of any specific vendor's piece(s) of hardware.

The use of functional/operational technical descriptions assists the person of skill in the art in understanding the described subject matter since, as is evident from the above discussion, one could easily, although not quickly, transcribe the technical descriptions set forth in this document as trillions of ones and zeros, billions of single lines of assembly-level machine code, millions of logic gates, thousands of gate arrays, or any number of intermediate levels of abstractions. However, if any such low-level technical descriptions were to replace the present technical description, a person of skill in the art could encounter undue difficulty in implementing the disclosure, because such a low-level technical description would likely add complexity without a corresponding benefit (e.g., by describing the subject matter utilizing the conventions of one or more vendor-specific pieces of hardware). Thus, the use of functional/operational technical descriptions assists those of skill in the art by separating the technical descriptions from the conventions of any vendor-specific piece of hardware.

In view of the foregoing, the logical operations/functions set forth in the present technical description are representative of static or sequenced specifications of various ordered-matter elements, in order that such specifications may be comprehensible to the human mind and adaptable to create many various hardware configurations. The logical operations/functions disclosed herein should be treated as such, and should not be disparagingly characterized as abstract ideas merely because the specifications they represent are presented in a manner that one of skill in the art can readily understand and apply in a manner independent of a specific vendor's hardware implementation.

At least a portion of the devices or processes described herein can be integrated into an information processing system. An information processing system generally includes one or more of a system unit housing, a video display device, memory, such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), or control systems including feedback loops and control motors (e.g., feedback for detecting position or velocity, control motors for moving or adjusting components or quantities). An information processing system can be implemented utilizing suitable commercially available components, such as those typically found in data computing/communication or network computing/communication systems.

The state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Various vehicles by which processes or systems or other technologies described herein can be effected (e.g., hardware, software, firmware, etc., in one or more machines or articles of manufacture), and that the preferred vehicle will vary with the context in which the processes, systems, other technologies, etc., are deployed. For example, if an implementer determines that speed and accuracy are paramount, the implementer may opt for a mainly hardware or firmware vehicle; alternatively, if flexibility is paramount, the implementer may opt for a mainly software implementation that is implemented in one or more machines or articles of manufacture; or, yet again alternatively, the implementer may opt for some combination of hardware, software, firmware, etc. in one or more machines or articles of manufacture. Hence, there are several possible vehicles by which the processes, devices, other technologies, etc., described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. In an embodiment, optical aspects of implementations will typically employ optically-oriented hardware, software, firmware, etc., in one or more machines or articles of manufacture.

The herein described subject matter sometimes illustrates different components contained within, or connected with, different other components. It is to be understood that such depicted architectures are merely examples, and that in fact, many other architectures can be implemented that achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably coupleable,” to each other to achieve the desired functionality. Specific examples of operably coupleable include, but are not limited to, physically mateable, physically interacting components, wirelessly interactable, wirelessly interacting components, logically interacting, logically interactable components, etc.

In an embodiment, one or more components may be referred to herein as “configured to,” “configurable to,” “operable/operative to,” “adapted/adaptable,” “able to,” “conformable/conformed to,” etc. Such terms (e.g., “configured to”) can generally encompass active-state components, or inactive-state components, or standby-state components, unless context requires otherwise.

The foregoing detailed description has set forth various embodiments of the devices or processes via the use of block diagrams, flowcharts, or examples. Insofar as such block diagrams, flowcharts, or examples contain one or more functions or operations, it will be understood by the reader that each function or operation within such block diagrams, flowcharts, or examples can be implemented, individually or collectively, by a wide range of hardware, software, firmware in one or more machines or articles of manufacture, or virtually any combination thereof. Further, the use of “Start,” “End,” or “Stop” blocks in the block diagrams is not intended to indicate a limitation on the beginning or end of any functions in the diagram. Such flowcharts or diagrams may be incorporated into other flowcharts or diagrams where additional functions are performed before or after the functions shown in the diagrams of this application. In an embodiment, several portions of the subject matter described herein is implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal-bearing medium used to actually carry out the distribution. Non-limiting examples of a signal-bearing medium include the following: a recordable type medium such as a floppy disk, a hard disk drive, a Compact Disc (CD), a Digital Video Disk (DVD), a digital tape, a computer memory, etc.; and a transmission type medium such as a digital or an analog communication medium (e.g., a fiber optic cable, a waveguide, a wired communications link, a wireless communication link (e.g., transmitter, receiver, transmission logic, reception logic, etc.), etc.).

While particular aspects of the present subject matter described herein have been shown and described, it will be apparent to the reader that, based upon the teachings herein, changes and modifications can be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Further, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense of the convention (e.g., “system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense of the convention (e.g., “a system having at least one of A, B, or C” would include but not be limited to systems that have A alone, B alone, C alone, A and B together, A and C together, B and C together, and/or A, B, and C together, etc.). Typically a disjunctive word or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, the operations recited therein generally may be performed in any order. Also, although various operational flows are presented in a sequence(s), it should be understood that the various operations may be performed in orders other than those that are illustrated, or may be performed concurrently. Examples of such alternate orderings includes overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

1. An electronic vaporizer device, comprising:
a cartridge assembly including one or more cartridges, each cartridge configured to hold multiple unit doses of an active agent;
a cannabinoid activation component;
at least one of a terpenoid activation component or a flavonoid activation component; and
a communication interface component.

2. (canceled)

3. The electronic vaporizer device of claim 1, wherein the cartridge assembly includes at least one cartridge having multiple unit doses of a solid plant extract composition including at least one of a cannabinoid, a terpenoid, or a flavonoid, or combinations or mixtures thereof.

4. (canceled)

5. (canceled)

6. The electronic vaporizer device of claim 1, wherein the cartridge assembly includes at least one cartridge having multiple unit doses of a vaporizable plant extract composition including at least one of a cannabinoid, a terpenoid, or a flavonoid.

7-15. (canceled)

16. The electronic vaporizer device of claim 1, further comprising:
an activation chamber operably coupled to the cannabinoid activation component and the at least one of the terpenoid activation component or the flavonoid activation component.

17. The electronic vaporizer device of claim 16, wherein the cannabinoid activation component is configured to selectively heat an interior environment within the activation chamber to a temperature and for a duration sufficient to cause the release of one or more cannabinoids from a unit dose received within the activation chamber.

18. (canceled)

19. The electronic vaporizer device of claim 16, wherein the cannabinoid activation component includes circuitry including at least one heating element configured to heat an interior environment within the activation chamber to a temperature ranging from about 120° C. to about 220° C.

20-24. (canceled)

25. The electronic vaporizer device of claim 16, wherein the electronic vaporizer device includes a terpenoid activation component; and wherein the terpenoid activation component is configured to selectively heat an interior environment within the activation chamber to a temperature and for a duration sufficient to cause the release of one or more terpenoids from a unit dose received within the activation chamber.

26. (canceled)

27. (canceled)

28. The electronic vaporizer device of claim 16, wherein the electronic vaporizer device includes a flavonoid activation component; and wherein the terpenoid activation component is configured to selectively heat an interior environment within the activation chamber to a temperature and for a duration sufficient to cause the release of one or more flavonoids from a unit dose received within the activation chamber.

29. The electronic vaporizer device of claim 28, wherein the flavonoid activation component includes circuitry configured to activate a target flavonoid dose profile.

30-41. (canceled)

42. The electronic vaporizer device of claim 1, wherein the communication interface component includes circuitry configured to exchange and store cannabis experience information from one or more of a cell phone device, a computer device, a desktop computer device, a laptop computer device, a managed node device, a notebook computer device, a remote controller, a tablet device, a wearable device, or an application interface with a smart device.

43-56. (canceled)

57. A vaporizing inhaler device, comprising
an aerosol generation assembly including one or more active agent reservoirs forming part of a modular structure;
a vaporization chamber; and
a cannabinoid activation component operably coupled to the vaporization chamber.

58. The vaporizing inhaler device of claim 57, further comprising:
a communication interface.

59. The vaporizing inhaler device of claim 57, further comprising:
at least one of a terpenoid activation component or a flavonoid activation component.

60-64. (canceled)

65. The vaporizing inhaler device of claim 57, wherein the cannabinoid activation component includes one or more heating elements.

66. (canceled)

67. The vaporizing inhaler device of claim 57, wherein the modular structure includes at least one of the one agent reservoir having multiple unit doses of a vaporizable composition including at least one of a cannabinoid, a terpenoid, or a flavonoid.

68-72. (canceled)

73. The vaporizing inhaler device of claim 57, further comprising:
a target experience component operably coupled to the aerosol generation assembly, the target experience component operable to generate one or more control commands for formulating a target aerosol composition having a target cannabinoid content, a target terpenoid content, or a target flavonoid content.

74. The vaporizing inhaler device of claim 57, further comprising:
a target experience component operably coupled to the aerosol generation assembly, the target experience component operable to generate one or more control commands for formulating a target aerosol composition having a target unit dose based on a pain mitigation profile or a stress mitigation profile.

75. The vaporizing inhaler device of claim 57, further comprising:
a target experience component operably coupled to the aerosol generation assembly, the target experience component operable to generate one or more control commands for formulating a target aerosol composition having a target unit dose based on an auto-immune disease profile.

76. The vaporizing inhaler device of claim 57, further comprising:
a target experience component operably coupled to the aerosol generation assembly, the target experience component operable to generate one or more control commands for formulating a target aerosol composition having a target unit dose based on a target cannabinoid/terpenoid/flavonoid profile.

77. The vaporizing inhaler device of claim 57, further comprising
a target experience component operably coupled to the aerosol generation assembly, the target experience component operable to generate one or more control commands for formulating a target aerosol composition having a target unit dose based on a cannabis experience profile.

78-80. (canceled)
